(19) United States
(12) Patent Application Publication
(10) Pub. No.: US 2008/0090306 A1
(43) Pub. Date: Apr. 17, 2008

(54) METHOD FOR IMMOBILIZING BIOMOLECULES

(75) Inventors: Koji KURUMA, Kanagawa (JP); Toshihide Ezoe, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/862,682

(22) Filed: Sep. 27, 2007

(30) Foreign Application Priority Data
Sep. 29, 2006 (JP) 267937/2006
Mar. 19, 2007 (JP) 070046/2007

(51) Int. Cl. G01N 33/543; G01N 21/55

(52) U.S. Cl. 436/518; 356/445

(57) ABSTRACT

An object of the present invention is to provide a method for preventing the generation of negative signals because of dissociation of a ligand from a surface and improving the precision of signals indicating binding between a ligand and an analyte. The present invention provides a method for immobilizing a biomolecule on a carrier having reactive groups which comprises: (i) a step of activating some reactive groups so that they can form a covalent bond with a biomolecule; (ii) a step of reacting the biomolecule with the aforementioned activated reactive groups; and (iii) a step of again activating some reactive groups; wherein the steps (i), (ii) and (iii) are performed in this order.

### TECHNICAL FIELD

The present invention relates to a method for immobilizing biomolecules and a method for analyzing interactions between biomolecules using the aforementioned method. In particular, the present invention relates to a method for immobilizing biomolecules that can be applied for a method for analyzing interactions between biomolecules with the use of a surface plasmon resonance biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

A measurement chip has been reported as a thin film having a functional group capable of immobilizing a physiologically active substance, on which a physiologically active substance is immobilized using a compound having a functional group binding to metal, a linker with a chain length of 10 or more atoms and a functional group capable of binding with the physiologically active substance (see JP Patent No. 2815120). Furthermore, a measurement chip comprising a metal film and a plasma polymerization film formed on the metal film has been reported (see JP Patent Publication (Kokai) No. 9-264843 A (1997)). When the surface of such measurement chip (biosensor) is produced, carboxylic acid amide is often formed in water (reaction of a polymer with a linker, followed by binding with a substance to be detected such as a protein). This reaction is generally performed by activating carboxylic acid using 1-(3-dimethylaminopropyl)-3 ethylcarbodiimide (EDC) which is a water-soluble carbodiimide, and N-hydroxysuccinimide (NHS), and then reacting the resultant with amine, so as to form carboxylic acid amide.

In general, a ligand may be insufficiently immobilized on a surface in an apparatus that is used for detecting the binding of an analyte to the ligand in the form of signals after immobilization of the ligand on the surface. In such case, negative signals are generated intermittently (also referred to as “negative drift”) because of dissociation of the ligand from the surface. This can affect observed results concerning the binding of the analyte, such that only signal amounts lower than an amount originally expected can be obtained. In particular, when a low-molecular-weight analyte that binds to a ligand protein is detected using an apparatus for detecting refractive-index variations in the near field as in the case of SPR, the signal amount depends on molecular weight. Hence, signals generated as a result of dissociation of one ligand molecule may be equivalent to binding signals of approximately 100 molecules of the analyte, so that negative binding signals are generated.

### DISCLOSURE OF THE INVENTION

The present invention is directed to addressing the above-mentioned problems of conventional techniques. Specifically, an object to be achieved by the present invention is to provide a method for preventing the generation of negative signals because of dissociation of a ligand from a surface and improving the precision of signals indicating binding between a ligand and an analyte.

As a result of intensive studies to achieve the above object, the present inventors have discovered that the generation of negative signals due to dissociation of a biomolecule (ligand) from a surface can be prevented by activating some reactive groups so that they can form covalent bonds with a biomolecule and so that the biomolecule is immobilized, and then again activating some reactive groups when the biomolecule is immobilized on a carrier having the reactive groups. The present inventors have further discovered that this will lead to improvement in the precision of signals (positive signals) that indicate binding between the biomolecule (ligand) and an analyte. Thus, the present inventors have completed the present invention.

Specifically, the present invention provides a method for immobilizing a biomolecule on a carrier having reactive groups which comprises:

(i) a step of activating some reactive groups so that they can form a covalent bond with a biomolecule;

(ii) a step of reacting the biomolecule with the aforementioned activated reactive groups; and

(iii) a step of again activating some reactive groups;

wherein the steps (i), (ii) and (iii) are performed in this order.

Preferably, the degree of activation performed in step (iii) is 0.01 to 0.5 times greater than that of activation performed in step (i).

Preferably, the reactive group is carboxyl group, amino group, or hydroxyl group.

Preferably, the reactive group is carboxyl group, and the step of activating reactive groups is a step of performing active esterification of the carboxyl group.

Preferably, the step of performing active esterification of carboxyl group comprises a step of activating carboxyl groups using carbodiimide, a derivative thereof, or salts of them, a nitrogen-containing compound, or a phenol derivative.

Preferably, the step of performing active esterification of carboxyl group comprises a step of activating carboxyl groups using a compound of any of the following formulas.

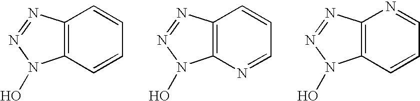

Preferably, the carrier having reactive groups is a carrier coated with a hydrophilic polymer having reactive groups.

Preferably, the hydrophilic polymer having reactive groups is polysaccharide.

Preferably, the hydrophilic polymer having reactive groups is immobilized on a metal film on the carrier via a self-assembled membrane composed of the formula A-1.
HS(CH₂)ₙX A-1
wherein n is an integer of 3 to 20, and X is a functional group.

Another aspect of the present invention provides a method for detecting or measuring a substance that interacts with a biomolecule, which comprises a step of causing a carrier having biomoecules immobilized thereon that is obtained according to the above method of the present invention to come into contact with a test substance.

Preferably, the substance that interacts with a biomolecule is detected or measured by a non-electrochemical method. Further preferably, the substance that interacts with a biomolecule is detected or measured by surface plasmon resonance analysis.

### BRIEF DESCRIPTION OF THE INVENTION

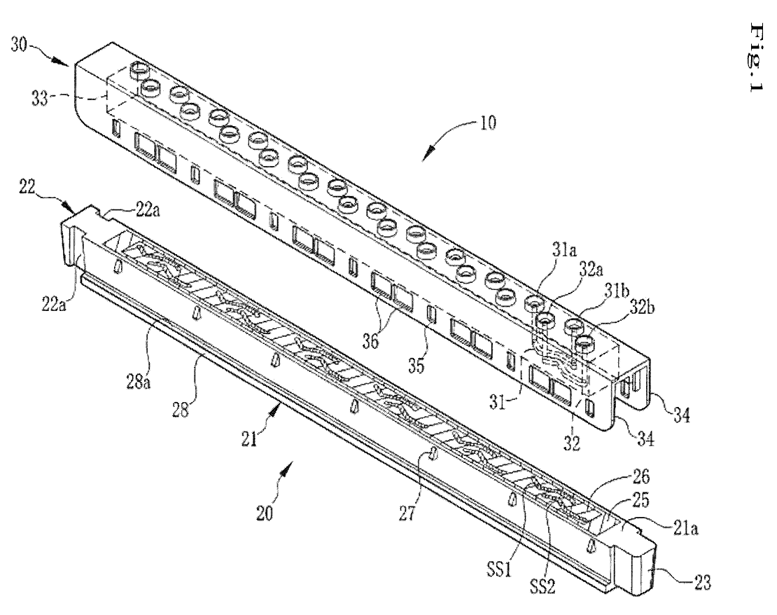

FIG. 1 shows an exploded perspective view of a sensor unit

### PREFERRED EMBODIMENTS OF THE INVENTION

Hereinafter, the embodiments of the present invention will be explained.

The method for immobilizing a biomolecule according to the present invention is a method for immobilizing a biomolecule on a carrier having reactive groups, which comprises:

(i) a step of activating some reactive groups so that they can form a covalent bond with a biomolecule;

(ii) a step of reacting the biomolecule with the aforementioned activated reactive groups; and

(iii) a step of again activating some reactive groups;

wherein the steps (i), (ii) and (iii) are performed in this order.

In this description, the expression “some reactive groups” refers to groups that are capable of reacting with a biomolecule after activation of reactive groups such that they can form covalent bonds with the biomolecule. For example, a case in which unactivated reactive groups are present is also included herein, as long as only some of reactive groups are activated when all reactive groups are activated. In addition, reactive groups in steps (i) and (iii) described above may be the same or differ from each other.

The expression “reactive groups” in the present invention refers to functional groups for binding with a biomolecule. Specific examples of such reactive groups include —COOH, —NR¹R² (wherein R¹ and R² denote mutually independently a hydrogen atom or a lower alkyl group), —OH, —SH, —CHO, —NR³NR¹R² (wherein R¹, R², and R³ denote mutually independently a hydrogen atom or a lower alkyl group), —NCO, —NCS, an epoxy group, or a vinyl group. The number of carbons in a lower alkyl group is not particularly limited herein. It generally ranges from approximately C1 to C10 and preferably ranges from approximately C1 to C6. Examples of preferable reactive groups include carboxyl groups, amino groups, and hydroxyl groups.

When the reactive groups are carboxyl groups, active esterification of carboxyl groups is preferable in the step of activating reactive groups. When the reactive groups are carboxyl groups, active esters are generated using a combination of carbodiimide (or a derivative thereof, a salt of carbodiimide, or a salt of a carbodiimide derivative) and N-hydroxysuccinimide, for example, and then covalent bonds can be formed with the amino groups of biomolecules.

Furthermore, when the reactive groups are amino groups, examples of a method that is often employed include a method that involves causing glutaraldehyde to act and then generating covalent bonds with amino groups of biomolecules, and a method that involves oxidizing biomolecules with periodate so as to cause direct covalent bonding with amino groups.

Moreover, when the reactive groups are hydroxyl groups, an example of a method that is often employed involves causing a polyepoxy compound or epichlorohydrin to act and then generating covalent bonds with amino groups of biomolecules. In addition, an example of a chemical reaction is a direct ether bond forming reaction using alkyl halide. However, application of such reaction to biomolecules can make the maintenance of physiological activity difficult.

As described above, a biosensor surface having carboxyl groups (carboxylic acid) is activated by a known method, specifically via 1-(3-Dimethylaminopropyl)-3 ethylcarbodiimide (EDC) which is water-soluble carbodiimide, and N-Hydroxysuccinimide (NHS), or via EDC alone, so that biomolecules having amino groups can be immobilized. Examples of other techniques for activation of carboxylic acid that can be used herein include: a method described in paragraph Nos. 0011 to 0022 of JP Patent Publication (Kokai) No. 2006-58071 A (JP Patent Application No. 2004-238396) (that is, a method for forming carboxylic amide groups through activation of carboxyl groups existing on a substrate surface using any one of compounds having specific structures, including an uronium salt, a phosphonium salt, and a triazine derivative); and a method described in paragraph Nos. 0011 to 0019 of JP Patent Publication (Kokai) No. 2006-90781 A (JP Patent Application No. 2004-275012) (that is, a method for forming carboxylic amide groups through activation of carboxyl groups existing on a substrate surface using a carbodiimide derivative or a salt thereof, esterification with the use of any one of compounds including a nitrogen-containing heteroaromatic compound having a hydroxyl group, a phenol derivative having an electron-withdrawing group, and an aromatic compound having a thiol group, and then reaction of the resultants with amine).

A method described in paragraph Nos. 0011 to 0019 of JP Patent Publication No. 2006-90781 (Kokai) A (JP Patent application No. 2004-275012) is explained as follows. A carbodiimide derivative or a salt thereof is preferably water soluble. Specifically, compounds 1 to 3 described in JP Patent Publication (Kokai) No. 2006-90781 A can be used herein, but the examples are not limited thereto.

Examples of types of nitrogen-containing heteroaromatic compound having a hydroxyl group are not particularly limited and include 5- or 7-membered saturated or unsaturated monocyclic rings and condensed rings containing at least one (e.g., 1, 2, or 3) nitrogen atom. Specific examples of such nitrogen-containing heteroaromatic compound having a hydroxyl group include compounds 4 to 12 described in JP Patent Publication (Kokai) No. 2006-90781 A, but are not limited thereto.

Specific examples of an electron-withdrawing group in a phenol derivative having an electron-withdrawing group include —NO₂, halogens (—F, —Cl, —Br, and —I), —S(CH₃)₂X⁻ (X denotes a monovalent anion (e.g., F⁻, Cl⁻, Br⁻, I⁻, At⁻, BF₄⁻, AsF₆⁻, PF₆⁻, SbF₆⁻, SbCl₆⁻, SnCl₆²⁻, FeCl₄⁻, BiCl₅²⁻, CF₃SO₂⁻, ClO₄⁻, FSO₂⁻, and F₂PO₂⁻)), —COOH, —CN, and —CHO. Furthermore, the σ value of an electron-withdrawing group is preferably 0.3 or higher. Further preferably the σ value of an electron-withdrawing group is 0.5 or higher and more preferably 0.7 or higher. σ values are outlined in the table attached with “Hammett's rule” (Naoki Inamoto, Maruzen Co., Ltd., 1983). Specific examples of a phenol derivative having an electron-withdrawing group include, but are not limited to, compounds 13 to 16 of JP Patent Publication (Kokai) No. 2006-90781 A.

Types of an aromatic compound having a thiol group (—SH) are not particularly limited and may be an aryl compound (e.g., benzene or naphthalene) or a heterocyclic compound containing at least one heteroatom. Examples of such heterocyclic compound include 5- or 7-membered saturated or unsaturated monocyclic rings or condensed rings containing one or more heteroatoms selected from the group consisting of nitrogen, oxygen, and sulfur atoms. Specific examples of such heterocyclic compound include pyridine, quinoline, isoquinoline, pyrimidine, pyrazine, pyridazine, phthalazine, triazine, furan, thiophene, pyrrole, oxazole, benzoxazole, thiazole, benzothiazole, imidazole, benzimidazole, thiadiazole, triazole, benzotriazole, 7-azabenzotriazole, and benzotriazine. Specific examples of an aromatic compound having an thiol group include, but are not limited to, compounds 17 to 19 described in JP Patent Publication (Kokai) No. 2006-90781 A.

The method for activating carboxyl groups existing on a carrier surface using a carbodiimide derivative or a salt thereof and then esterifying the carboxyl groups with any one of compounds including a nitrogen-containing heteroaromatic compound having a hydroxyl group, a phenol derivative having an electron-withdrawing group, and an aromatic compound having a thiol group is not particularly limited. Such activation and esterification can be performed by a conventional method known by persons skilled in the art. Specifically, a solution (e.g., an aqueous solution) containing a carbodiimide derivative or a salt thereof and any one of compounds including a nitrogen-containing heteroaromatic compound having a hydroxyl group, a phenol derivative having an electron-withdrawing group, and an aromatic compound having a thiol group is caused to come into contact with a carrier having carboxyl groups on its surface. Thus, esterification can be performed.

Compounds 1 to 3 and compounds 4 to 19 described above in JP Patent Publication (Kokai) No. 2006-90781 A are known compounds and can be synthesized by conventional methods. Alternatively, commercially available products can also be used. Specifically, compounds 1 to 19 are marketed by Kokusan Chemical Co., Ltd., Aldrich, K. Sakai & Co., Ltd., or Dojindo Laboratories, for example, or they can be synthesized by methods described in literature (Bull. Chem. Soc. Jpn., 60, 2409 (1987)., J. Polym. Sci., Polym. Chem. Ed., 17, 2013 (1979), and J. Polym. Sci., Polym. Chem. Ed., 16, 475 (1978)).

In the present invention, carboxylic amide groups are formed by reacting amine with the above-formed active esters. Methods for performing reaction with amine are not particularly limited and can be performed by conventional methods known by persons skilled in the art. For example, such reaction can be performed by causing an ethanolamine-HCl solution to come into contact with a carrier.

Moreover, another method for activation of carboxyl groups is a method that uses a nitrogen-containing compound. Specifically, a nitrogen-containing compound represented by the following formula (Ia) or (Ib) [wherein R₁ and R₂ mutually independently denote a carbonyl group, a carbon atom, or a nitrogen atom, which may have a substituent, R₁ and R₂ may form 5- to 6-membered rings via binding, “A” denotes a carbon atom or a phosphorus atom, which has a substituent, “M” denotes an (n−1)-valent element, and “X” denotes a halogen atom] can also be used.

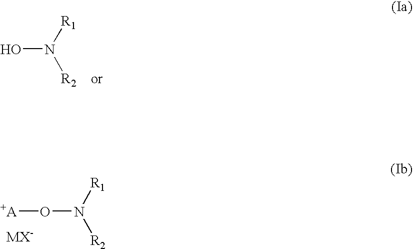

R₁ and R₂ mutually independently denote a carbonyl group, a carbon atom, or a nitrogen atom, which may have a substituent, and preferably R₁ and R₂ form 5- to 6-membered rings via binding. Particularly preferably, hydroxysuccinic acid, hydroxyphthalic acid, 1-hydroxybenzotriazole, 3,4-dihydroxy-3-hydroxy-4-oxo-1,2,3-benzotriazine, and a derivative thereof are provided.

Further preferably, a nitrogen-containing compound represented by the following compound can also be used.

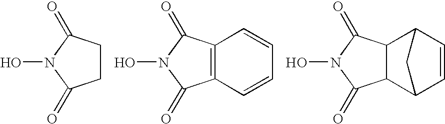

More preferably, a compound represented by the following formula (II) [wherein “Y” and “Z” mutually independently denote CH or a nitrogen atom] can also be used as a nitrogen-containing compound.

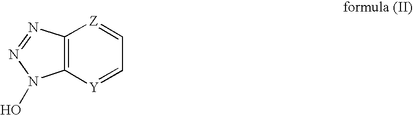

Specifically, the following compounds can be used, for example.

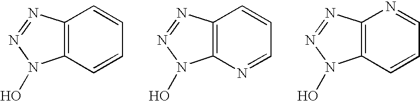

Further preferably, the following compound can also be used as a nitrogen-containing compound.

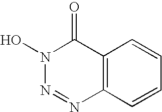

Further preferably, a compound represented by the following formula (III) [wherein “A” denotes a carbon atom or a phosphorus atom, which has a substituent, “Y” and “Z” mutually independently denote CH or a nitrogen atom, “M” denotes a (n−1)-valent element, and X denotes a halogen atom] can also be used as a nitrogen-containing compound.

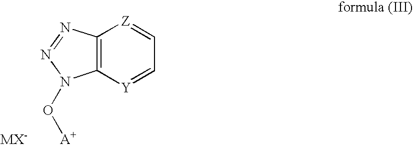

A substituent of a carbon atom or a phosphorus atom denoted by “A” is preferably an amino group having a substituent. Furthermore, a dialkylamino group such as a dimethylamino group or a pyrrolidino group is preferable. Examples of an (n−1)-valent element denoted by “M” include a phosphorus atom, a boron atom, and an arsenic atom. A preferable example of such (n−1)-valent element is a phosphorus atom. Examples of a halogen atom denoted by “X” include a fluorine atom, a chlorine atom, a bromine atom, and an iodine atom. A preferable example of such halogen atom is a fluorine atom.

Furthermore, specific examples of such nitrogen-containing compound represented by formula (III) include the following compounds, for example.

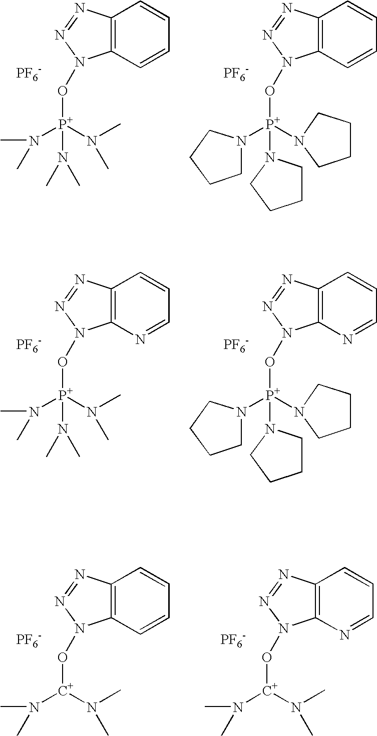

Further preferably, a compound represented by the following formula (IV) [wherein “A” denotes a carbon atom or a phosphorus atom, which has a substituent, “M” denotes an (n−1)-valent element, and “X” denotes a halogen atom] can also be used as a nitrogen-containing compound.

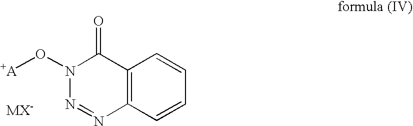

Specifically, the following compound can be used, for example.

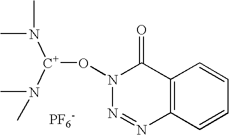

Moreover, as a method for activating carboxyl groups, the use of a phenol derivative having an electron-withdrawing group is also preferable. Furthermore, the σ value of the electron-withdrawing group is preferably 0.3 or higher. Specifically, the following compounds or the like can also be used.

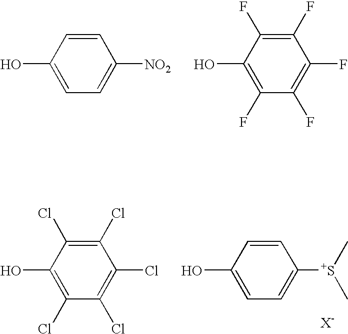

Furthermore, a carbodiimide derivative can further be used separately for such method for activating carboxyl groups in combination with the above compounds. Preferably, a water-soluble carbodiimide derivative can be used in combination with such compounds. Further preferably, the following compound (1-Ethyl-3-(3-dimethylaminopropyl)carbodiimide, hydrochloride) can be used in combination with such compounds.

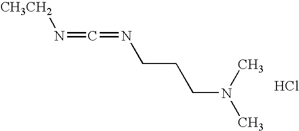

The above carbodiimide derivative and nitrogen-containing compound or phenol derivative can be used not only in such manner, but also independently, if desired. Preferably, a carbodiimide derivative and a nitrogen-containing compound are used in combination.

Furthermore, the following compound can also be used in such method for activating carboxyl groups. The compound can be used independently and can also be used in combination with a carbodiimide derivative, a nitrogen-containing compound, and/or a phenol derivative.

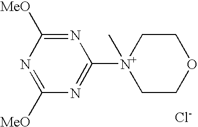

In the present invention, the above-mentioned step of activating reactive groups is performed before and after the step of reacting a biomolecule with reactive groups. Specifically, in the present invention, some reactive groups are activated, so that they can form covalent bonds with the biomolecule, the biomolecule is then reacted with the thus activated reactive groups, and then some reactive groups are further activated again.

In the present invention, the product of [concentration of activated compound]×[time for activation] is defined as [degree of activation]. The degree of activation observed for the second and subsequent activations (after immobilization of a ligand) in the present invention are each preferably 0.001 times or more and 1 time or less, further preferably 0.005 times or more and 0.8 times or less, and most preferably 0.01 times or more and 0.5 times or less than the degree observed for the first activation (before immobilization of the ligand).

In the present invention, the reactive group which is introduced into a polymer as a group for immobilizing biomolecules may be a functional group such as carboxylic group or amino group, as well as biomolecules such as biotin-binding protein (avidin, streptavidin, NeutraAvidin and the like), Protein A, Protein G, antigen, antibody (for example, known tag antibody such as anti-GST antibody). Such biomolecules may be previously immobilized, and then another biomolecules as mentioned below can be immobilized on said biomolecules. Further, biomolecules having membrane structure such as lipid can be immobilized by using an immobilizing layer obtained by introducing alkane into a polymer. Further, various protein can be handled by controlling the length of polymer chain, the thickness of polymer, the density of polymer, or the amount of reactive groups which are introduced into polymer, depending on the use. Further, His-tag legand and the like can be immobilized via metal chelate by introducing NTA (nitrilotriacetic acid) and the like into a polymer as an immobilizing group.

The carrier used in the present invention is preferably a metal surface or metal film. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above carrier, an interstitial layer consisting of chrome or the like may be provided between the carrier and a metal layer.

The film thickness of a metal film is not limited. When the metal film is used for a surface plasmon resonance biosensor, the thickness is preferably between 0.1 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and have excellent workability are preferably used.

The aforementioned carrier is immobilized on the dielectric block of a measurement unit and is unified therewith to construct a measurement chip. This measurement chip may be exchangeably formed. An example will be given below.

FIG. 1 is an exploded perspective view of a sensor unit 10 used in a measurement that utilizes SPR. The sensor unit 10 is composed of a total internal reflection prism (optical block) 20 that is a transparent dielectric body and a flow channel member 30 equipped on the total internal reflection prism 20. The flow channel member 30 has two types of flow channels, namely, a first flow channel 31 located on the back side of the figure and a second flow channel 32 located on the front side of the figure. When the sensor unit 10 is used in measurement, the two types of flow channels 31 and 32 are used in combination to measure a single sample. However, the details will be described later. In the flow channel member 30, six flow channels 31 and six flow channels 32 are established in the longitudinal direction, so that six samples can be measured in a single sensor unit 10. It is to be noted that the number of either the flow channel 31 or 32 is not limited to six, but that it may be 5 or less, or 7 or more.

The total internal reflection prism 20 is composed of a prism main body 21 formed in a long trapezoidal shape, a gripper 22 established at one end of the prism main body 21, and a projecting portion 23 established at the other end of the prism main body 21. This total internal reflection prism 20 is molded by extrusion molding, for example. The prism main body 21, the gripper 22, and the projecting portion 23 are integrally molded.

The prism main body 21 has a substantially trapezoidal longitudinal section wherein the lower base is longer than the upper base. Light irradiated from the lateral side of the bottom is gathered to an upper surface 21a. A metal film (thin film layer) 25 for exciting SPR is established on the upper surface 21a of the prism main body 21. The shape of the metal film 25 is rectangular such that it faces the flow channels 31 and 32 of the flow channel member 30. The metal film 25 is molded by an evaporation method, for example. The Metal film 25 is made of gold, silver, or the like, and the thickness thereof is 50 nm, for example. The thickness of the metal film 25 is selected as appropriate, depending on the material of the metal film 25, the wavelength of light irradiated during the measurement, etc.

On the metal film 25, a layer 26 for immobilizing a physiologically active substance is established. The layer 26 which comprises a hydrophobic polymer or a hydrophilic polymer, has a binding group for immobilizing a physiologically active substance. A physiologically active substance is immobilized on the metal film 25 via the layer 26 comprising a hydrophobic polymer or a hydrophilic polymer.

The carrier having reactive groups according to the present invention is preferably a carrier coated with a hydrophilic polymer having reactive groups. Such hydrophilic polymer having reactive groups can be bound to the above-described metal surface or metal film directly or via an intermediate layer.

Examples of a hydrophilic polymer compound that is used in the present invention include polysaccharides (e.g., agarose, dextran, carrageenan, alginic acid, starch, and cellulose) and synthetic polymer compounds (e.g., polyvinyl alcohol). In the present invention, a polysaccharide is preferably used, and dextran is most preferably used.

In the present invention, preferably, a hydrophilic polymer with an average molecular weight of 10,000 to 2,000,000 can be used. Preferably, a hydrophilic polymer with an average molecular weight of 20,000 to 2,000,000, further preferably 30,000 to 1,000,000, and most preferably 200,000 to 800,000 can be used.

A hydrophilic polymer used in the present invention preferably has a thickness between 1 and 300 nm in an aqueous solution. If the film thickness is too small, the quantities of biomolecules immobilized are reduced. In addition, since a hydration layer on the sensor surface becomes thin, biomolecules denature by themselves, and it thereby becomes difficult to detect the interaction of such biomolecules with test substances. On the other hand, if the film thickness is too large, it impairs dispersions of test substances in the film. Moreover, in particular, when such interaction is detected from the side opposite to the surface of a sensor substrate on which a hydrophilic polymer is immobilized, the distance between a detection surface and an interaction-forming portion is increased, resulting in a decrease in detection sensitivity. The film thickness of a hydrophilic polymer in an aqueous solution can be evaluated by AFM, ellipsometry measurement, etc.

A polyhydroxy compound that is an example of the hydrophilic polymer can be carboxylated by reacting the compound with bromoacetic acid under basic conditions, for example. Through control of the reaction conditions, a given proportion of hydroxy groups contained in the polyhydroxy compound in its initial state can be carboxylated. In the present invention, 1% to 90% of hydroxy groups can be carboxylated, for example. In addition, the degree of carboxylation (% carboxylation) on a surface coated with an arbitrary polyhydroxy polymer compound can be calculated by the following method, for example. The film surface is subjected to gas-phase modification at 50° C. for 16 hours using di-tert-butyl carbodiimide/pyridine catalyst and trifluoroethanol. A fluorine amount derived from trifluoroethanol is measured via ESCA (electron spectroscopy for chemical analysis). The ratio of the fluorine amount to an oxygen amount on the film surface (hereinafter, referred to as an F/O value) is then calculated. The theoretical F/O value when all the hydroxy groups are carboxylated is determined to represent 100% carboxylation. The F/O value resulting when carboxylation is performed under arbitrary conditions is measured and then the degree of carboxylation (% carboxylation) at this time can be calculated.

In the present invention, a hydrophilic polymer having reactive groups can be bound directly to a metal surface or a metal film or indirectly via an intermediate layer. As such “intermediate layer” used herein, a layer comprising a hydrophobic polymer compound or a self-assembled membrane can be used, for example. Hereinafter, the hydrophobic polymer compound and the self-assembled membrane will be explained.

The hydrophobic polymer compound is a polymer compound lacking water-absorbing properties and having solubility (25° C.) in water of 10% or less, more preferably 1% or less, and most preferably, 0.1% or less.

Hydrophobic monomers that form such hydrophobic polymer compound can be arbitrarily selected from among vinyl esters, acrylic acid esters, methacrylic acid esters, olefins, styrenes, crotonic acid esters, itaconic acid diesters, maleic acid diesters, fumaric acid diesters, allyl compounds, vinyl ethers, vinyl ketones, and the like. Such hydrophobic polymer compound may be a homopolymer comprising one type of monomer, or a copolymer comprising two or more types of monomer.

Examples of such hydrophobic polymer compound that is preferably used in the present invention include polystyrene, polyethylene, polypropylene, polyethylene terephthalate, polyvinylchloride, polymethyl methacrylate, polyester, and nylon.

A carrier can be coated with such hydrophobic polymer compound by a conventional method such as spin coating, air-knife coating, bar coating, blade coating, slide coating, or curtain coating. Coating can also be performed by a spray method, an evaporation method, a cast method, a dipping method, or the like.

The coating thickness of the hydrophobic polymer compound is not particularly limited and is preferably 0.1 nm to 500 nm, and particularly preferably 1 nm to 300 nm.

Next, the self-assembled membrane will be explained. Sulfur compounds such as thiol and disulfides are spontaneously adsorbed onto a noble metal (e.g., gold) substrate, so that a monomolecular-sized ultra-thin film can be produced. A cluster of such sulfur compounds has a sequence depending on the crystal lattice of a substrate or the molecular structure of the adsorbed molecules. Hence, the thus provided membrane is referred to as a self-assembled membrane. Specifically, in the present invention, a hydrophilic polymer can be adhered to a metal film via an organic molecule X¹—R¹—Y¹. The organic molecule X¹—R¹—Y¹ will be described in detail.

X¹ is a group having binding affinity for a metal film. Specifically, asymmetric or symmetric sulfide (—SSR¹¹Y¹¹, —SSR¹Y¹), sulfide (—SR¹¹Y¹¹, —SR¹Y¹), diselenide (—SeSeR¹¹Y¹¹, —SeSeR¹Y¹), selenide (SeR¹¹Y¹¹, —SeR¹Y¹), thiol (—SH), nitrile (—CN), isonitrile, nitro (—NO₂), selenol (—SeH), a trivalent phosphorus compound, isothiocyanate, xanthate, thiocarbamate, phosphine, thio acid, or dithioic acid (—COSH, —CSSH) is preferably used.

R¹ (and R¹¹) is occasionally interrupted via hetero atoms, is preferably linear (unbranched) because of appropriately dense packing, and is occasionally a hydrocarbon chain containing double and/or triple bonds. The carbon chain can be occasionally perfluorinated.

Y¹ and Y¹¹ are groups for binding with a polyhydroxy polymer compound. Y¹ and Y¹¹ are preferably the same and are capable of binding directly with such polyhydroxy polymer compound or binding with the same after activation. Specifically, a hydroxyl, carboxyl, amino, aldehyde, hydrazide, carbonyl, epoxy, or vinyl group can be used, for example.

In the present invention, 7-carboxy-1-heptanethiol, 10-carboxy-1-decanethiol, 4,4′-dithiodibutyric acid, 1′-hydroxy-1-undecanethiol, 11-amino-1-undecanethiol, and the like can be used as self-assembled compounds, for example.

In the present invention, an alkanethiol derivative represented by the formula A-1 (in the formula A-1, n represents an integer of 3 to 20, and X represents a functional group) is used as a self-assembled membrane-forming compound to thereby form a monolayer having orientation in a self-assembled manner on the basis of the Au—S bond and the van der Waals force between the alkyl chains. The self-assembled membrane is produced by a quite convenient approach wherein the gold substrate is dipped in a solution of the alkanethiol derivative. The carrier can be bound with a hydrophilic polymer compound by forming a self-assembled membrane by use of a compound represented by the formula A-1 wherein X is a functional group which can bind to the hydrophilic polymer compound.
HS(CH_{2+L)}ₙX A-1

When an amino group is used as a group for binding a hydrophilic polymer compound in the present invention, an alkanethiol having an amino group at the end may be a compound comprising a thiol group and an amino group linked via an alkyl chain (formula A-2) (in the formula A-2, n represents an integer of 3 to 20), or may be a compound obtained by reaction between alkanethiol having a carboxyl group at the end (formula A-3 or A-4) (in the formula A-3, n represents an integer of 3 to 20, and in the formula A-4, n each independently represents an integer of 1 to 20) and a large excess of hydrazide or diamine. The reaction between alkanethiol having a carboxyl group at the end and a large excess of hydrazide or diamine may be performed in a solution state. Alternatively, the alkanethiol having a carboxyl group at the end may be bound to the carrier surface and then reacted with a large excess of hydrazide or diamine.
HS(CH₂)ₙNH₂ A-2
HS(CH₂)ₙCOOH A-3
HS(CH₂)ₙ(OCH₂CH₂)ₙOCH₂COOH A-4

The repeating number of alkyl group of the formulas A-2 to A-4 is preferably 3 to 20, more preferably 3 to 16, and most preferably 4 to 8. If the alkyl chain is short, formation of self-assembled membrane becomes difficult, and if the alkyl chain is long, water solubility decreases and the handling becomes difficult.

Any compound may be used as the diamine used in the present invention. An aqueous diamine is preferable for use in the biosensor surface. Specific examples of the aqueous diamine may include aliphatic diamine such as ethylenediamine, tetraethylenediamine, octamethylenediamine, decamethylenediamine, piperazine, triethylenediamine, diethylenetriamine, triethylenetetraamine, dihexamethylenetriamine, and 1,4-diaminocyclohexane, and aromatic diamine such as paraphenylenediamine, metaphenylenediamine, paraxylylenediamine, metaxylylenediamine, 4,4′-diaminobiphenyl, 4,4′-diaminodiphenylmethane, 4,4′-diaminodiphenylketone, and 4,4′-diaminodiphenylsulfonic acid. From the viewpoint of increasing the hydrophilicity of the biosensor surface, a compound comprising two amino groups linked via an ethylene glycol unit (formula A-5) may also be used. The diamine used in the present invention is preferably ethylenediamine or the compound represented by the formula A-5 (in the formula A-5, n and m each independently represent an integer of 1 to 20), more preferably ethylenediamine or 1,2-bis(aminoethoxy)ethane (represented by the formula A-5 wherein n=2 and m=1).
H₂N(CH₂)ₙ(OCH₂CH₂)ₘO(CH₂)ₙNH₂ A-5

The alkanethiol may form a self-assembled membrane by itself or may form a self-assembled membrane by mixing it with another alkanethiol. It is preferred for use in the biosensor surface that a compound capable of suppressing the nonspecific adsorption of a physiologically active substance should be used as the another alkanethiol. The aforementioned Professor Whitesides et al. have investigated in detail self-assembled membrane capable of suppressing the nonspecific adsorption of a physiologically active substance and have reported that a self-assembled membrane formed from alkanethiol having a hydrophilic group is effective for suppressing nonspecific adsorption (Langmuir, 17, 2841-2850, 5605-5620, and 6336-6343 (2001)). In the present invention, any of compounds described in the aforementioned papers may be used preferably as the alkanethiol that forms a mixed monolayer. In terms of excellent ability to suppress nonspecific adsorption and ease of acquisition, it is preferred that alkanethiol having a hydroxyl group (formula A-6) or alkanethiol having an ethylene glycol unit (formula A-7) (in the formula A-6, n represents an integer of 3 to 20, and in the formula A-7, n and m each independently represent an integer of 1 to 20) should be used as the alkanethiol that forms a mixed monolayer.
HS(CH₂)ₙOH A-6
HS(CH₂)ₙ(OCH₂CH₂)ₘOH A-7

When several types of alkane thiol are mixed to form a self-assembled membrane, the repeating number of alkyl group of the formulas A-2 to A-4 is preferably 4 to 20, more preferably 4 to 16, and most preferably 4 to 10. Further, the repeating number of alkyl group of the formulas A-6 and A-7 is preferably 3 to 16, more preferably 5 to 12, and most preferably 5 to 10.

A biomolecule immobilized on the carrier in the present invention is not particularly limited, as long as it interacts with a measurement target. Examples thereof may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against 0 antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When the biomolecule is a protein such as enzyme or a nucleic acid, immobilization thereof can be carried out by binding it to a reactive group on a carrier by the use of amino group, thiol group and the like of the biomolecule.

Preferably, these biomolecules can be immobilized by coating a solution containing the biomolecule on a carrier on which a hydrophilic polymer compound is immobilized and then drying it.

With regard to the concentration of a solution (an applied solution) that contains biomolecules, it is preferable that the concentration of biomolecules immobilized on a carrier surface be high. The aforementioned concentration is preferably between 0.1 mg/ml and 10 mg/ml, and more preferably between 1 mg/ml and 10 mg/ml, although it depends on the type of biomolecules.

In the process of drying such a solution that contains biomolecules, the biomolecules tend to be precipitated from the peripheral portion of the applied solution, or from a portion in which the liquid remains immediately before the applied solution is dried. Thereby, the quantities of biomolecules immobilized on the carrier surface vary. This is not preferable. In order to uniform the quantities of biomolecules immobilized on the carrier surface, it is preferable that the viscosity of the applied solution be set at high, so far as it does not inhibit the binding of the biomolecules to the carrier surface. By setting the viscosity of the applied solution at high, the movement of the biomolecules contained in the applied solution in the horizontal direction towards the carrier surface can be suppressed during the drying process. As a result, variations in the quantities of biomolecules immobilized can be suppressed. The viscosity of the applied solution is preferably maintained at 0.9 cP or more during the drying process.

The term “drying process” is used in the present invention to mean natural drying whereby after application of a solution that contains biomolecules, the solution is left at rest, or an intentional drying process whereby the speed of drying the aforementioned solution is increased by heating or air-blowing. An increase in the speed of drying the solution (the applied solution) that contains biomolecules is effective for suppressing variations in the quantities of biomolecules immobilized. The drying speed is increased, and the drying process is terminated at a speed that is sufficiently faster than the speed of biomolecules that move in the horizontal direction. Thereby, the drying process can be terminated before the substantial movement of such biomolecules, so that variations in the quantities of biomolecules immobilized can be suppressed. The type of a method of increasing such a drying speed is not particularly limited. Examples of such a method include a method of increasing the temperature of the applied solution or a temperature in dry environment, a method of adding evaporation energy by irradiation with infrared ray or laser, a method of decreasing a solvent vapor pressure during the drying process by air blowing or the like, and a method of enlarging an evaporation area with respect to the amount of the applied solution by applying the solution to form a thin layer. In particular, when such an applied solution contains water, a drying speed is increased by performing the drying process in an environment wherein there is a great difference between a dry-bulb temperature and a wet-bulb temperature. The drying process is carried out in an environment wherein the difference between such a dry-bulb temperature and a wet-bulb temperature is preferably 7° C. or more, more preferably 10° C. or more, and further more preferably 13.5° C. or more. In addition, taking into consideration a production process, the time required for the drying process is preferably 10 minutes or less, more preferably 5 minutes or less, and particularly preferably 1 minute or less.

An example of a method of applying a solution that contains biomolecules is a method particularly using a dispenser that quantitatively discharges a solution to be applied. The discharge port of the dispenser is moved on a carrier at a certain speed at certain intervals, so that the solution can be uniformly applied at any given sites on the carrier. When the solution is applied using a dispenser, the interval between the carrier and the discharge port is extremely narrowed, and the thickness of the applied solution is reduced, so that the thickness of biomolecules can be uniformed. Further, the drying speed can also be increased. Thus, the use of such a dispenser is preferable. Another preferred method of applying a solution that contains biomolecules is spin coating. This method is particularly preferably applied when the thickness of the applied film is reduced. In this method, since the drying process is carried out after a solution having a uniformed thickness has been formed, it is preferable to prevent evaporation of the solution during rotation of a spin coater. If a method of placing a carrier in a hermetically sealed vessel or the like during rotation is applied to maintain the concentration of a solvent existing around the carrier at high, the drying speed can be controlled before and after formation of a thin film during rotation. Thus, this method is particularly preferable. After completion of such an application process, the applied solution is preferably dried under conditions wherein temperature and humidity are kept constant.

When the interaction of biomolecules with test substances is detected, variations in the quantities of biomolecules immobilized on the sensor surface cause an error in quantitative and kinetic evaluation of such an interaction. In order to keep such an error to a minimum, the quantities of biomolecules immobilized are preferably uniformed. A CV value (coefficient variation) (standard deviation/mean value), which indicates variations in the quantities of biomolecules immobilized on the surface of a carrier used in detection of an interaction, is preferably 15% or less, and more preferably 10% or less. Such a CV value can be calculated based on the quantities of biomolecules immobilized on at least two sites, preferably 10 or more sites, and more preferably 100 or more sites on the carrier surface. Uniformity can be evaluated by quantifying the quantities of substances existing on a sensor carrier before and after immobilization of biomolecules. However, such uniformity can also be evaluated by fluorescently-labeling substances that have been known to bind to biomolecules, immobilizing such fluorescently-labeled substances on a sensor carrier, and then measuring fluorescence intensity using a fluorescence microscope or the like. Moreover, it is also possible to quantify biomolecules using an SPR imager, an ellipsometer, TOF-SIMS, an ATR-IR apparatus, etc.

In the present invention, a compound having a residue capable of forming hydrogen bond (hereinafter referred to as Compound S) may be used for improving storage stability of biomolecules which was immobilized on a carrier.

Generally, biomolecules such as protein maintain its three-dimensional structure by coordination of water molecules in a solution, but when it is dried, biomolecule cannot maintain its three-dimensional structure and is denatured. Further, biomolecule is contained in a hydrophicil polymer compound on a surface of carrier, biomolecules aggregate by drying, and aggregates are produced. The compound S having a residue capable of forming hydrogen bond which may be used in the present invention can be used for the purpose of suppressing denature of biomolecules by maintaining the three-dimensional structure in place of water or suppressing the aggregation by steric effect by covering the biomolecule.

In the present invention, the Compound S having a residue capable of forming hydrogen bond is preferably added as a aqueous solution to a layer on carrier where biomolecule was immobilized. Compound S can be added by coating a mixed solution of Compound S and biomolecule on a surface of carrier, or by immobilizing biomolecules on a surface of carrier and then over-coating the Compound S. When a mixed solution of Compound S and biomolecule is coated, fluctuation of the amount of immobilized biomolecules can be reduced. Preferably, an aqueous solution of Compound S can be added to carrier in a state of thin film. A method for forming thin film on carrier may be any known method. Examples thereof include extrusion coating, curtain coating, casting, screen printing, spin coating, spray coating, slidebead coating, slit and spin coating, slit coating, die coating, dip coating, knife coating, blade coating, flow coating, roll coating, wire-bar coating, and transferring printing. In the present invention, spray coating or spin coating is preferably used, and spin coating is more preferably used as a method for forming a thin film on carrier, since a coated film having a controlled film thickness can be easily prepared.

The concentration of the applied solution of compound S is not particularly limited, as long as it does not cause a problem regarding permeation into a layer that contains biomolecules. The aforementioned concentration is preferably between 0.1% by weight and 5% by weight. In addition, in terms of applicability and regulation of pH, a surfactant, a buffer, an organic solvent, a salt may also be added to the applied solution.

The compound S having a residue capable of forming hydrogen bond is preferably a compound which is non-volatile under normal pressure at normal temperature. The average molecular weight of the compound is preferably 350 to 5,000,000, more preferably 1,200 to 2,000,000, most preferably 1,200 to 70,000. The compound S having a hydroxyl group in molecule is preferably saccharide. The saccharide may be monosaccharide or polysaccharide. In case of n-saccharide, n is preferably 4 to 1,200, and n is more preferably 20 to 600.

If the mean molecular weight of compound S is too low, the compound is crystallized on the surface of a carrier. This causes disruption of a hydrophilic polymer layer, on which biomolecules are immobilized, and disruption of the three-dimensional structure of the biomolecules. In contrast, if the mean molecular weight of compound S is too high, it causes problems such that it impairs immobilization of biomolecules on a carrier, that a layer that contains biomolecules cannot be impregnated with compound S, and that layer separation occurs.

For the purpose of suppressing degradation of biomolecules immobilized on a carrier, the aforementioned compound S having a residue capable of forming hydrogen bond preferably has a dextran skeleton or a polyethylene oxide skeleton. The type of a substituent used is not limited, as long as the object of the present invention can be achieved. Moreover, for the purpose of suppressing degradation of biomolecules immobilized on a carrier, a nonionic compound having no dissociable groups is preferably used as compound S. Furthermore, the aforementioned compound S having a residue capable of forming a hydrogen bond preferably has high affinity for water molecules. A distribution coefficient LogP value between water and n-octanol is preferably 1 or greater. Such LogP value can be measured by the method described in Japanese Industrial Standard (JIS), Z7260-107 (2000), “Measurement of distribution coefficient (1-octanol/water)—Shaking method,” etc.

Specific examples of compound S having a residue capable of forming hydrogen bond include: compounds consisting of two or more types of residues selected from polyalcohols such as polyvinyl alcohol, proteins such as collagen, gelatin, or albumin, polysaccharides such as hyaluronic acid, chitin, chitosan, starch, cellulose, alginic acid, or dextran, polyethers including polyethyleneoxy-polypropylene oxide condensates such as polyethylene glycol, polyethylene oxide, polypropylene glycol, polypropylene oxide, or Pluronic, Tween 20, Tween 40, Tween 60, Tween 80, etc.; and derivatives and polymers of such compounds. Of these, polysaccharides and polyethers are preferable, and polysaccharides are more preferable. Specifically, dextran, cellulose, Tween 20, Tween 40, Tween 60, and Tween 80 are preferably used. Further, a nonvolatile monomer and a nonvolatile water-soluble oligomer described in JP Patent Publication (Kokai) No. 2006-170832 A can also be used. Examples of such a nonvolatile monomer used herein may include: tetrose, pentose, heptose and hextose, wherein a hydroxyl group may be protected by a protecting group, and their glycoside; methyl glucoside; and cyclitols, wherein a hydroxyl group may be protected by a protecting group. Moreover, examples of such a nonvolatile water-soluble oligomer include: an oligomer represented by general formula (1) (—[CH₂—CH(CONH₂)-]n-), general formula (2) (—[CH₂—CH₂—O-]n-), or general formula (3) (—[CH₂—CH(OH)-]n-) (wherein, in general formulas (1) to (3), n represents an integer between 10 and 200); and an oligosaccharide having an n number of sugars (2≦n≦10), wherein a hydroxyl group may be protected by a protecting group. Furthermore, sugars described in US 2003/0175827 and DE 20306476A1, such as trehalose, sucrose, maltose, lactose, xylitol, fructose, mannitol, glucose, xylol, maltodextran, saccharose, or polyvinylpyrrolidone may also be used. It is preferable that such compound S be substantially identical to the basic skeleton of a hydrophilic polymer used in the present invention. The term “basic skeleton” is used herein to mean a ring structure of sugar, for example. Although the type of a functional group or length differs, if such a ring structure is identical, it is considered that the basic skeleton is substantially identical.

With regard to the content of compound S having a residue capable of forming hydrogen bond existing on a carrier, the ratio of the mean molecular weight of the aforementioned compound S to the mean molecular weight of a hydrophilic polymer is preferably between 0.005 and 0.2. If such a ratio is lower than the aforementioned range, compound S is likely to be crystallized. If such a ratio is higher than the aforementioned range, it is difficult for compound S to permeate into a hydrophilic polymer layer. When the aforementioned ratio is set within the aforementioned range, such problems are solved. Thereby, a higher effect of suppressing denature of biomolecules and a higher effect of suppressing aggregation can be obtained.

A carrier on which a biomolecule has been immobilized according to the method of the present invention can be used for detecting and/or measuring a substance interacting with the biomolecule. Specifically, according to the present invention, a method for detecting or measuring a substance that interacts with a biomolecule is provided, which comprises a step of causing the carrier (obtained according to the method of the present invention through immobilization of a biomolecule thereon) to come into contact with a test substance.

A sample containing a substance that interacts with the above-described biomolecule can be used as such test substance, for example.

The carrier on which biomolecules are immobilized by the method of the present invention can be used as a biosensor or a part thereof. The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

In the present invention, it is preferable to detect and/or measure an interaction between a physiologically active substance immobilized on the substrate for sensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443, paragraph 0011). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (OSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (OSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (OSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (OSP) is measured periodically when a certain period of time has elapsed.

If the test substance contained in the sample solution is bound to the sensing substance, the refractive index of the sensing substance is changed by this binding over time. Accordingly, the above attenuated total reflection angle (OSP) is measured periodically after the elapse of a certain time, and it is determined whether or not a change has occurred in the above attenuated total reflection angle (OSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (OSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (OSP) has changed. Accordingly, based on the amount by which the derivative value has changed, a binding state between a sensing substance and a test substance can be measured (Japanese Patent Publication (Kokai) No. 2003-172694 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

The biosensor of the present invention can be used as a biosensor, which has a waveguide structure on the surface of a carrier, for example, and which detects refractive index changes using such a waveguide. In this case, the waveguide structure on the carrier surface has a diffraction grating, and in some cases, an additional layer. This waveguide structure is a planar waveguide body comprising a thin dielectric layer. Light gathered to the waveguide body form is introduced into such a thin layer by total internal reflection. The transmission velocity of the thus introduced light wave (hereinafter referred to as “mode”) is indicated as a C/N value. Herein, C indicates the velocity of light in a vacuum, and N indicates an effective refractive index of the mode introduced into the waveguide body. Such an effective refractive index N is determined based on the structure of the waveguide body on one face, and is determined based on the refractive index of a medium adjacent to the thin waveguide layer on the other face. Conduction of a light wave is carried out not only in a thin planar layer, but also by another waveguide structure, and in particular, by a stripped waveguide body. In such a case, the waveguide structure is processed into the shape of a stripped film. It is an important factor for a biosensor that changes in effective refractive indexes N are generated as a result of changes in the medium adjacent to the waveguide layer, and changes in the refractive index and thickness of the waveguide layer itself or an additional layer adjacent to the waveguide layer.

The structure of a biosensor of this system is described in page 4, line 48 to page 14, line 15, and FIGS. 1 to 8 of JP Patent Publication (Kokoku) No. 6-27703 B (1994), and column 6, line 31 to column 7, line 47, and FIGS. 9A and 9B of U.S. Pat. No. 6,829,073.

For example, in one embodiment, there is a structure whereby a waveguide layer comprising a planar thin layer is established on a substrate (e.g. Pyrex (registered trademark) glass). A waveguide layer and a substrate form together a so-called waveguide body. Such a waveguide layer can be a multilayer laminated body such as an oxide layer (SiO2, SnO2, Ta2O5, TiO2, TiO2-SiO2, HfO2, ZrO2, Al2O₃, Si3N4, HfON, SiON, scandium oxide, or a mixture thereof) or a plastic layer (e.g. polystyrene, polyethylene, polycarbonate, etc.). For transmission of light into a waveguide layer as a result of total internal reflection, the refractive index of the waveguide layer must be greater than that of the adjacent medium (for example, a substrate, or an additional layer as described later). A diffraction grating is disposed on the surface of the waveguide layer or in the bosom thereof, which faces to a substrate or a measured substance. Such a diffraction grating can be formed in a carrier according to embossing, holography, or other methods. Subsequently, the upper surface of the diffraction grating is coated with a thin waveguide film having a higher refractive index. The diffraction grating has the functions to focus rays of light incident on the waveguide layer, to discharge the mode already introduced into the waveguide layer, or to transmit a portion of the mode in the travel direction and reflect a portion thereof. The grating area of the waveguide layer is covered with an additional layer. Such an additional layer can be a multilayer film, as necessary. This additional layer is able to have the function to carry out selective detection of a substance contained in a measured substance. In a preferred embodiment, a layer having a detection function can be established on the outermost surface of such an additional layer. As such a layer having a detection function, a layer capable of immobilizing biomolecules can be used.

In another embodiment, it is also possible to adopt a structure whereby an array of diffraction grating waveguides is incorporated into wells of a microplate (JP Patent Publication (Kohyo) No. 2007-501432 A). That is to say, if such diffraction grating waveguides are aligned in the form of an array at the bottoms of wells of a microplate, the screening of a drug or chemical substance can be carried out at a high throughput.

In order to detect biomolecules existing on the upper layer (detection area) of a diffraction grating waveguide, the diffraction grating waveguide detects incident light and reflected light, so as to detect changes in refractive properties. For this purpose, one or more light sources (e.g. laser or diode) and one or more detectors (e.g. a spectrometer, a CCD camera, or other light detectors) can be used. As a method of measuring changes in refractive indexes, there are two different operational modes, namely, spectroscopy and an angle method. In spectroscopy, broadband beam used as incident light is transmitted to a diffraction grating waveguide, and reflected light is gathered, followed by a measurement with a spectrometer, for example. By observing the spectrum position of a resonant wavelength (peak), changes in refractive indexes on the surface of the diffraction grating waveguide or a periphery thereof, namely, a bond can be measured. On the other hand, in an angle method, light of a nominally single wavelength is gathered such that it generates a certain range of irradiation angle, and it is directed into the diffraction grating waveguide. The reflected light is measured with a CCD camera or other types of light detectors. By measuring the position of a resonance angle reflected by the diffraction grating wavelength, changes in refractive indexes on the surface of the diffraction grating waveguide or a periphery thereof, namely, a bond can be measured.

The present invention will be further specifically described in the following examples. However, the examples are not intended to limit the scope of the present invention.

### EXAMPLES

### Example 1

A hydrogel membrane on which protein can be immobilized was prepared using an SAM compound with high water solubility. The membrane was evaluated in terms of the amount of protein immobilized and non-specific adsorption properties.

(1) Preparation of Substrate

A 1 mM aqueous solution of 6-Amino-1-octanethiol, hydrochloride (produced by Dojindo Laboratories) was prepared. The solution was designated solution A.

Next, a gold thin film was formed on the top face of a plastic prism obtained via injection molding of ZEONEX (produced by Zeon Corporation) by the following method.

A substrate holder of a sputtering apparatus was installed with a prism. After vacuuming (base pressure of 1×10⁻³ Pa or less), Ar gas (1 Pa) was introduced. RF power (0.5 kW) was applied to the substrate holder for approximately 9 minutes, while rotating (20 rpm) the substrate holder. Thus, the prism surface was subjected to plasma treatment. Next, Ar gas flow was stopped, vacuuming was performed, and then Ar gas was introduced again (0.5 Pa). DC power (0.2 kW) was applied to an 8-inch Cr target for approximately 30 seconds, while rotating (10 rpm to 40 rpm) the substrate holder. Thus, a 2-nm Cr thin film was formed. Next, Ar gas flow was stopped, vacuuming was performed again, and then Ar gas was introduced again (0.5 Pa). DC power (1 kW) was applied to an 8-inch Au target for approximately 50 seconds, while rotating (20 rpm) the substrate holder. Thus, an approximately 50-nm Au thin film was formed.

The above-obtained sensor stick (B) on which Au thin film had been formed was immersed in solution A at 40° C. for 1 hour, and then washed 5 times with ultra-pure water.

(2) Active Esterification of CMD (carboxymethyldextran)

10 g of a 1% by weight CMD (produced by Meito Sangyo Co., Ltd.: molecular weight of 1,000,000, substitution degree of 0.65) solution was dissolved. 10 ml of a mixed aqueous solution of 0.02 M EDC (1-Ethyl-3-[3-Dimethylaminopropyl]carbodiimide Hydrochloride) and 87.5 mM HOBt (1-Hydroxybenzotriazole) was added. The solution was stirred at room temperature for 1 minute and then allowed to stand for 1 hour.

(3) Binding Reaction of CMD to Substrate

1 ml of the CMD solution prepared via active esterification in (2) was added dropwise onto the substrate prepared in (1). Spin coating was performed at 1000 rpm for 45 seconds, so that the carboxymethyldextran thin film subjected to active esterification was formed on the substrate having amino groups. After 15 minutes of reaction at room temperature, the resultant was immersed in a 1 N NaOH aqueous solution for 30 minutes and then washed 5 times with ultra pure water. Thus, a CMD-immobilized substrate was obtained.

(4) Binding Reaction of NeutrAvidin to CMD-Immobilized Substrate

The substrate prepared in (3) was set in a surface plasmon resonance apparatus. A mixed aqueous solution of 0.2 M EDC and 0.05 M NHS (N-Hydroxysuccinimide) was added onto the substrate, and then the substrate was allowed to stand for 7 minutes. The substrate surface was washed with Acetate 5.0 (produced by BIACORE) and then air was blown over the surface. An acetate 5.0 solution of 0.2 mg/ml NeutrAvidin (produced by PIERCE) was added onto the substrate surface. The substrate surface was then allowed to stand for 15 minutes. The substrate surface was washed with 1 M Ethanolamine aqueous solution (adjusted at pH 8.5) and then allowed to stand for 7 minutes. The surface was further washed with 1×PBS (pH 7.4), so that a substrate upon which NeutrAvidin had been immobilized was obtained.

Signals generated before and after immobilization of NeutrAvidin were measured using a surface plasmon resonance apparatus. The amount of NeutrAvidin immobilized was 15000 RV on average. Here, a difference in terms of angle of resonance per DMSO 1% is represented by 1500 RV.

(5) Activation of NeutrAvidin-immobilized substrate

A mixed aqueous solution of 0.2 M EDC and 0.05 M NHS was added onto the NeutrAvidin-immobilized substrate prepared in (4) and then the substrate was allowed to stand for different lengths of time as listed in Table 1. The substrate surface was washed with 1×PBS (pH 7.4).

### Comparative Example 1

### Washing with NaOH Aqueous Solution

A 10 mM NaOH aqueous solution was added onto the NeutrAvidin-immobilized substrate prepared in (4) and then the substrate was allowed to stand for different lengths of time as listed in Table 1. The substrate surface was washed with 1×PBS (pH 7.4).

### Comparative Example 2

### Washing with HCl Aqueous Solution

A 100 mM HCl aqueous solution was added onto the NeutrAvidin-immobilized substrate prepared in (4), and then the substrate was allowed to stand for different lengths of time as listed in 1. The substrate surface was washed with 1×PBS (pH 7.4).

### Example 2

### Binding Reaction of Biotin to Neutravidin-Immobilized Substrate

Example 2 relates to the binding of biotin to sensor samples obtained in Example 1, Comparative example 1, and Comparative example 2.

Samples prepared in Example 1, Comparative example 1, and Comparative example 2 were set in a surface plasmon resonance apparatus. 1×PBS (pH 7.4) was injected and then the resultant was allowed to stand for 10 minutes. After confirmation of the baseline (the angle of resonance after 10 minutes was determined to be a reference point), a 1×PBS (pH 7.4) solution of 0.2 mg/ml biotin was injected. The resultant was allowed to stand for 30 minutes. Thereafter, 1×PBS (pH 7.4) was injected. The resultant was allowed to stand for 1 minute. The difference between the angle of resonance at this time and the angle of resonance at the original point was determined to indicate the amount of biotin bound.

Table 1 shows the baseline fluctuation and the amount of biotin bound for each sample.

Whereas the surfaces in the Comparative examples were unable to suppress the occurrence of negative drifts because of the detachment of NeutrAvidin or caused the loss of NeutrAvidin activity, the surfaces of the present invention could successfully suppress the detachment of NeutrAvidin while keeping NeutrAvidin activity.

TABLE 1

| | Treatment after | | | Baseline | Amount of | |
|---|---|---|---|---|---|---|
| | immobilization | | | fluc- | Biotin | |
| | of NeutrAvidin | | | tuation | bound | |
| No. | Reagent | Time | | (RV) | (RV) | Remarks |
| 1 | NaOH | 5 | minutes | 120 | −30 | Comparative |
| | | | | | | example |
| 2 | NaOH | 10 | minutes | 90 | −20 | Comparative |
| | | | | | | example |
| 3 | NaOH | 30 | minutes | 40 | −30 | Comparative |
| | | | | | | example |
| 4 | HCl | 5 | minutes | 40 | −35 | Comparative |
| | | | | | | example |
| 5 | HCl | 10 | minutes | 20 | −20 | Comparative |
| | | | | | | example |
| 6 | HCl | 30 | minutes | 5 | −5 | Comparative |
| | | | | | | example |
| 7 | EDC/NHS | 30 | seconds | 5 | 125 | Present |
| | | | | | | invention |
| 8 | EDC/NHS | 1 | minute | 5 | 120 | Present |
| | | | | | | invention |
| 9 | EDC/NHS | 3 | minutes | 4 | 120 | Present |
| | | | | | | invention |

| | | | | | |
|---|---|---|---|---|---|
| 10 | None | — | 180 | −60 | Comparative |
| | | | | | example |

### EFFECT OF THE INVENTION

According to the present invention, the generation of negative signals due to dissociation of a ligand from a surface can be prevented, so that the precision of signals indicating the binding between a biomolecule (ligand) and an analyte can be improved. In the present invention, when activation is performed to an excessive extent after immobilization of a biological substance (e.g., protein), the binding sites of the immobilized biological substance (e.g., protein) with a test substance may be directly and/or indirectly blocked. Accordingly, in the present invention, optimum conditions for activation after immobilization are preferably activation conditions that depend on the properties of the biological substance and the amount of the same immobilized but cause activation weaker than that before immobilization (lower concentration of a compound to be activated/shorter time for activation).

1. A method for immobilizing a biomolecule on a carrier having reactive groups which comprises:
(i) a step of activating some reactive groups so that they can form a covalent bond with a biomolecule;
(ii) a step of reacting the biomolecule with the aforementioned activated reactive groups; and
(iii) a step of again activating some reactive groups;
wherein the steps (i), (ii) and (iii) are performed in this order.

2. The method of claim 1 wherein the degree of activation performed in step (iii) is 0.01 to 0.5 times greater than that of activation performed in step (i).

3. The method of claim 1 wherein the reactive group is carboxyl group, amino group, or hydroxyl group.

4. The method of claim 1 wherein the reactive group is carboxyl group, and the step of activating reactive groups is a step of performing active esterification of the carboxyl group.

5. The method of claim 4 wherein the step of performing active esterification of carboxyl group comprises a step of activating carboxyl groups using carbodiimide, a derivative thereof, or salts of them, a nitrogen-containing compound, or a phenol derivative.

6. The method of claim 4 wherein the step of performing active esterification of carboxyl group comprises a step of activating carboxyl groups using a compound of any of the following formulas.

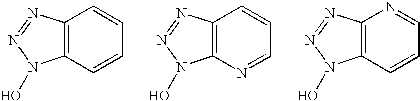

7. The method of claim 1 wherein the carrier having reactive groups is a carrier coated with a hydrophilic polymer having reactive groups.

8. The method of claim 7 wherein the hydrophilic polymer having reactive groups is polysaccharide.

9. The method of claim 7 wherein the hydrophilic polymer having reactive groups is immobilized on a metal film on the carrier via a self-assembled membrane composed of the formula A-1.
HS(CH₂)ₙX A-1
wherein n is an integer of 3 to 20, and X is a functional group.

10. A method for detecting or measuring a substance that interacts with a biomolecule, which comprises a step of causing a carrier having biomoecules immobilized thereon that is obtained according to the method of claim 1 to come into contact with a test substance.

11. The method of claim 10 wherein the substance that interacts with a biomolecule is detected or measured by a non-electrochemical method.

12. The method of claim 10 wherein the substance that interacts with a biomolecule is detected or measured by surface plasmon resonance analysis.
